(19) United States
(12) Patent Application Publication
(10) Pub. No.: US 2025/0031982 A1
(43) Pub. Date: Jan. 30, 2025

(54) ESTIMATION OF BLOOD PRESSURE IN THE HEART

(71) Applicants: OSLO UNIVERSITETSSYKEHUS HF, Oslo (NO); KING'S COLLEGE LONDON, London (GB)

(72) Inventors: Otto SMISETH, Oslo (NO); Joao Filipe FERNANDES, London (GB)

(21) Appl. No.: 18/702,419

(22) PCT Filed: Oct. 20, 2022
(86) PCT No.: PCT/EP2022/079293

§ 371 Date: Apr. 18, 2024

(30) Foreign Application Priority Data
Oct. 21, 2021 (GB) 2115123.8

(51) Int. Cl. A61B 5/021; A61B 5/00; A61B 5/055; A61B 6/50; A61B 8/04; A61K 31/216; A61K 31/41; A61K 31/7048

(52) U.S. Cl. CPC A61B 5/021; A61B 5/4839; A61B 5/7278; A61K 31/216; A61K 31/41; A61K 31/7048; A61B 5/055; A61B 6/503; A61B 8/04

(57) ABSTRACT

A system and method for estimating LV filling pressure of the heart of a human patient. The system includes a data processing apparatus, an imaging system for non-invasively obtaining images of the patient's heart and for providing imaging data to the data processing apparatus, and a data input device for receiving patient specific parameters relating to BMI and systolic arterial blood pressure of the patient and for providing the patient specific parameters to the data processing apparatus. The data processing apparatus is configured to perform the method including the steps of using the imaging data and the patient specific parameters, determining an estimate of the minimum LV diastolic pressure, estimating the peak pressure drop and obtaining a maximum difference between the LA pressure and LV pressure, and calculating an estimate of the LV filling pressure.

### TECHNICAL FIELD

The present invention relates to the estimation of blood pressure inside the heart, and in particular to a system and method for estimation of filling pressure in the left side of the heart using non-invasive imaging data.

### BACKGROUND OF THE INVENTION

Heart failure is one of the most prevalent heart diseases. In about one half of all heart failure patients, left ventricular (LV) pump function is normal when measured as LV ejection fraction. This condition is named heart failure with preserved ejection fraction, abbreviated as HFpEF. In this large group of heart failure patients, the assessment of cardiac function is challenging and there is a need for better methods to determine blood pressure inside the heart.

A clear indicator of HFpEF is obtained by the demonstration of elevated LV filling pressure, which is done in the prior art via a pressure catheter inside the left ventricle or indirectly by a catheter in the right ventricle and the pulmonary artery. These examinations are referred to as heart catheterization, and are done in hospitals, usually only when planning heart surgery or cardiac interventions.

If LV filling pressure could be measured without the need for invasive techniques needing a hospital visit, the doctor could more easily confirm the HFpEF diagnosis and hence prescribe drugs to reduce LV filling pressure earlier, thereby preventing hospitalizations. It was recently demonstrated that patients with HFpEF can be treated successfully with empagliflozin, which is an oral medication. Entresto is now indicated to reduce the risk of cardiovascular death and hospitalization for heart failure in adult patients with chronic heart failure, i.e. also covering HFpEF.

### SUMMARY OF THE INVENTION

Viewed from a first aspect, the present invention provides a system for estimating the LV filling pressure of a human patient, the system comprising:
a data processing apparatus;
an imaging system for non-invasively obtaining images of the patient's heart and for providing imaging data to the data processing apparatus; and
a data input device for receiving patient specific parameters relating to body mass index (BMI) and systolic blood pressure of the patient and for providing the patient specific parameters to the data processing apparatus;
wherein the data processing apparatus is configured to:
(a) use the imaging data to obtain cardiac markers, including an estimate of LA reservoir strain and an estimate of the time constant of LV isovolumic pressure decay, and use the patient specific parameters directly or to determine additional cardiac markers including systolic pressure and BMI;
(b) determine an estimate of the minimum LV diastolic pressure as a summation of a value derived from each of the cardiac markers multiplied by a corresponding constant of proportionality and added to a constant value, wherein the constant value and the various constants of proportionality have been derived from a statistical analysis of historic patient data for a plurality of patients;
(c) estimate the peak pressure drop during early diastole between left atrium and left ventricle and hence obtaining a maximum difference between the LA pressure and LV pressure; and
(d) calculate an estimate of LV filling pressure as the sum of values from step (b) and step (c) above.

The system of the first aspect is considered to provide an accurate and reliable non-invasive method for assessment of LV filling pressure, which can then be used to improve diagnosis of heart failure. The estimates of LV filling pressure can be applied in HFpEF to improve a diagnosis. Furthermore, the estimates of LV filling pressure can be used in both HFpEF and in heart failure with reduced ejection fraction to determine need for therapy to reduce symptoms caused by high filling pressure.

The measurement of LV filling pressure can be done as left atrial (LA) mean pressure, as LV diastolic pressure prior to LA contraction (LV pre-A pressure), or as LV pressure at the end of diastole (LV end-diastolic pressure). LA mean pressure and LV pre-A pressure have essentially similar value, whereas LV end-diastolic pressure is in most cases slightly higher. In this document, the term LV filling pressure refers collectively to LA mean pressure and LV pre-A pressure. Estimation of LV end-diastolic pressure is also described and implemented in this document.

The estimate of LV filling pressure provided by the claimed system can be used for assessing the condition of the patient's heart, and hence to provide a medical practitioner with information useful to make the HFpEF diagnosis. Alternative diagnoses typically need to be excluded before it can be concluded that a patient suffers from HFpEF. In relation to other disorders the demonstration of elevated LV filling pressure may be used as one of several criteria for the diagnosis or as indication that further diagnostic tests are needed.

The ability of the proposed method to estimate LV filling pressure in patients with atrial fibrillation, severe valvular heart disease and some other conditions may require modification of the algorithm with inclusion of other imaging-based parameters in the algorithm.

In step (a) the patient specific parameters may be used to determine other markers including BMI and/or systolic pressure. Alternatively or additionally the patient specific parameters may be used directly. For example, the patient specific parameters may include patient height and/or weight with these being used directly rather than being converted into a BMI value. The systolic pressure may be used along with the original patient specific parameters (e.g. height and/or weight) or along with BMI.

The system may include an output device for providing an indication of the estimated LV filling pressure, such as a display device for visual feedback to a user or a data transfer device for communicating data to another system, such as an external computer system or network, or a mobile device. The data processing apparatus may be configured to compare the estimate of the LV filling pressure to a threshold value, such as a value considered to identify healthy or unhealthy patients. In that case an output device may be configured to indicate to the user if the patient is healthy or unhealthy, in particular with regard to an elevated LV filling pressure. The patient may be considered to potentially suffer from HFpEF, i.e. possible stiffening of the heart muscle, if the estimate of the LV filling pressure is equal to or in excess of 15 mmHg, or optionally the threshold value may be set lower, for example in excess of 12 mmHg.

The summation at step (b) may be of the magnitude of the cardiac markers multiplied by the associated constant. Alternatively step (b) may use some other value derived from the magnitude of the cardiac marker, e.g. a square thereof. The system makes use of a constant value and various constants of proportionality that have been derived from a statistical analysis of historic patient data for a plurality of patients. The statistical analysis may for example comprise a multivariate linear regression analysis accounting for the different cardiac markers. Alternatively, the statistical analysis may be based on non-linear analysis, multiple logistic regression, discriminant analysis, or artificial neural networks, including physics-informed neural networks (PINNs), amongst others. The inventors have found that by making appropriate use of a suitably comprehensive dataset of a given patient population it is possible to estimate the minimum LV pressure via a statistical analysis, such as the multivariate linear regression described in more detail herein or alternative techniques based on the same cardiac markers.

As well as the markers discussed above, the cardiac markers used in the statistical analysis may also include other measures and/or estimates, such as one or more of: end systolic or end diastolic LV volume normalized to Body mass index (BMI) or body surface area (respectively, ESVi or EDVi); the ratio between peak early diastolic velocity and peak late diastolic velocity across mitral valve (EoverA); maximal mitral annular tissue velocity at early diastole filling (e′) and/or LA volume indexed to body surface area (LAVi).

The systolic pressure, which can refer to arterial, aortic or LV systolic pressure, is a peak system pressure and can be measured via suitable means, such as via a blood pressure cuff. The system can receive this measure via the data input device, e.g. as a numeric value. BMI can be obtained in known ways, i.e. from height and weight measurements and once again the system can receive this measure via the data input device. That may be done as the source height and weight data, or as input of a pre-calculated BMI. The imaging data is used to obtain an estimate of LA reservoir strain and an estimate of the time constant of LV isovolumic pressure decay. In prior art invasive methods this is done according to known principles documented with pressure catheter inside the left ventricle, but in this method we use a non-invasive imaging based method to obtain the time constant of LV isovolumic pressure decay. The same imaging data may also be used for the estimation of peak pressure drop at step (c).

The LA reservoir strain may be any suitable indicator of strain in this context, such as a long axis reservoir strain, a short axis reservoir strain, a mean area strain or a volumetric measure of strain.

The summation of step (b) may be considered as:

$$A + B \times ( systolic aortic pressure ) + C \times ( BMI ) + D \times ( estimate of LA reservoir strain ) + E \times ( estimate of the time constant of LV isovolumic pressure decay )$$

where each of A, B, C, D and E are constants obtained via the statistical analysis. Optionally, there may be further terms in the sum for ESVi, EDVi, EoverA, e′, LV mass and/or LAVi as mentioned above, each with their own constant of proportionality.

In one example, the constant A is in the range −24.1 to 0.3, for example it may be in the range −14.4 to −6.5. Optionally it is about −6.9, for example −6.885.

In one example, the constant B, i.e. the constant of proportionality associated with the systolic artic pressure (peak system pressure) is in the range 0.001 to 0.056, for example it may be in the range 0.02 to 0.03. Optionally it is about 0.025.

In one example, the constant C, i.e. the constant of proportionality associated with the BMI is in the range −0.085 to 0.432, for example it may be in the range 0.10 to 0.30. Optionally it is about 0.243.

In one example, the constant D, i.e. the constant of proportionality associated with the estimate of LA reservoir strain is in the range −0.126 to 0.040, for example it may be in the range −0.10 to 0.03. Optionally it is about −0.0973.

In one example, the constant E, i.e. the constant of proportionality associated with the estimate of the time constant of LV isovolumic pressure decay is in the range 0.097 to 0.275, for example it may be in the range 0.12 to 0.20. Optionally it is about 0.1575.

Thus, in one particular example the summation of step (b) may be expressed as:

$$- 6.885 + 0.025 \times ( systolic pressure ) + 0.243 \times ( BMI ) - 0.0973 \times ( estimate of LA reservoir strain ) + 0.1575 \times ( estimate of the time constant of LV isovolumic pressure decay )$$

Where one or more of ESVi, EDVi, EoverA, e′, and/or LAVi are included then the relevant constants may be in the ranges tabulated below:

TABLE 1
Constant ranges for the potential inclusion of further
cardiac markers in the statistical analysis.

| Cardiac marker | Minimum | Maximum |
|---|---|---|
| ESVi | −0.049 | 0.046 |
| EDVi | −0.034 | 0.052 |
| EoverA | −2.828 | 1.658 |
| e′ | −0.172 | 0.724 |
| LAVi | −0.106 | 0.091 |

The data processing apparatus may be configured to provide an estimated LV diastolic pressure curve showing estimated pressure throughout diastole. This may be done by obtaining key markers for LV pressure in relation to time and/or pressure for points in the cycle and fitting a normalised curve shape to the key markers. The key markers for the estimated LV pressure curve will include the estimated LV filling pressure as well as one or more of: time of aortic valve closure; time of mitral valve (MV) opening; minimum LV pressure and respective time; time of peak E-wave; time of end diastasis, time of peak A-wave, and time of end diastole (corresponding to MV closure). In addition, the pressure may be estimated at some or all of these time points, such that the system may be configured to provide a non-invasive estimate of at least seven patient-specific pressure-time points thus enabling close fitting of a normalised curve. The normalised curve may be an average of previously obtained sample patient data. The data processing apparatus may be configured to interpolate curve segments of the normalised curve in order to fit to the key markers of pressure and time to thereby obtain the estimated LV pressure curve. Furthermore, the increase in LV pressure caused by LA contraction will be calculated from the estimated LA to LV pressure difference and estimated diastolic stiffness of the left ventricle.

The imaging system is for non-invasively obtaining images of the patient's heart and may be, for example, an ultrasound imaging system, a magnetic resonance imaging (MRI) system, an x-ray imaging system such as computed tomography (CT), or any other system able to obtain suitable images of the heart. In example embodiments echocardiography is used and hence the imaging system may comprise an ultrasound probe and associated computer devices as are already known for use in echocardiography.

The data processing apparatus may comprise a computer processor and may for example be a computer device. It may include a self-contained computer device, such as a computer system provided with the imaging system for controlling the imaging system and/or for image processing functions. Alternatively or additionally, the computer device may comprise multiple distributed computer processor devices within a network, including data storage and/or computer processing within a cloud network.

The data input device may be any suitable device for receiving the data from a user, such as by entry of numeric information, and transmitting it to the data processing apparatus. It may be provided by one or more parts of the data processing apparatus or a peripheral thereof, such as a keyboard, touch screen, or other user interface. The data input device may be provided by a mobile computing device such as a smartphone or tablet.

Viewed from a second aspect, the present invention provides a computer programme product comprising instructions, which when executed on a data processing apparatus will configure the data processing apparatus to receive imaging data from an imaging system; receive patient specific parameters relating to BMI and systolic blood pressure of the patient; and then:
(a) use the imaging data to obtain cardiac markers including an estimate of LA reservoir strain and an estimate of the time constant of LV isovolumic pressure decay, and use the patient specific parameters directly or to determine additional cardiac markers including systolic pressure and BMI;
(b) determine an estimate of the minimum LV diastolic pressure as a summation of a value derived from each of the cardiac markers multiplied by a corresponding constant of proportionality and added to a constant value, wherein the constant value and the various constants of proportionality have been derived from a statistical analysis of historic patient data for a plurality of patients;
(c) estimate the peak pressure drop during early diastole between left atrium and left ventricle and hence obtaining a maximum difference between the LA pressure and LV pressure; and
(d) calculate an estimate of the LV filling pressure as the sum of values from step (b) and step (c) above.

The second aspect may be considered as a computer programme product with instructions that when executed will configure a data processing device to carry out the method of the third aspect, as discussed below. The computer programme device may also include any of the further features set out below and/or may configure the data processing device to perform as discussed above in connection with the first aspect and optional features thereof. The computer programme product may be provided for a computer system of an imaging system, such as the imaging system of the first aspect.

Viewed from a third aspect the invention provides a method of estimating the LV filling pressure of the heart of a human patient, the method comprising:
(a) using non-invasive imaging and other non-invasive measurement of the patient, determining cardiac markers including: systolic pressure, BMI or other patent specific parameters such as height and weight, an estimate of LA reservoir strain and an estimate of the time constant of LV isovolumic pressure decay;
(b) determining an estimate of the minimum LV diastolic pressure as a summation of a value derived from each of the cardiac markers multiplied by a corresponding constant of proportionality in addition to a constant value, wherein the constant value and the various constants of proportionality have been derived from statistical analysis of historic patient data for a plurality of patient (c) estimating the peak pressure drop during early diastole between left atrium and left ventricle and hence obtaining a maximum difference between the LA pressure and LV pressure; and
(d) calculating an estimate of the LV filling pressure as the sum of values from step (b) and step (c) above.

The estimate of the LV filling pressure may be used for assessing the condition of the patient's heart, and hence the method may provide a medical practitioner with information useful to make the HFpEF diagnosis as mentioned above. The demonstration of elevated LV filling pressure may be used as one of several criteria for the diagnosis or as indication that further diagnostic tests are needed.

The method for assessment of the LV filling pressure can thus be used to improve diagnosis of heart failure. The estimates of LV filling pressure may be applied in HFpEF to improve a diagnosis. Furthermore the estimates of LV filling pressure can be used in both HFpEF and in heart failure with reduced ejection fraction to determine need for therapy to reduce symptoms caused by high filling pressure.

The method may involve use of any or all features of the system discussed above. For example, the method may include using an output device for providing an indication of the estimated LV filling pressure, such as using a display device for visual feedback to a user or a data transfer device for communicating data to another system, such as an external computer system or network, or a mobile device.

The method may include comparing the estimate of the LV filling pressure to a threshold value, such as a value considered to identify healthy or unhealthy patients. This may be done via the data processing apparatus for example. In that case an output device may be used to indicate to the user if the patient is healthy or unhealthy, in particular with regard to an elevated LV filling pressure. The patient may be considered to potentially suffer from HFpEF, i.e. possible stiffening of the heart muscle, if the estimate of the LV filling pressure is equal to or in excess of 15 mmHg, or optionally the threshold value may be set lower, for example in excess of 12 mmHg.

The summation at step (b) may be of the magnitude of the cardiac markers multiplied by the associated constant. Alternatively, it may use some other value derived from the magnitude of the cardiac marker, e.g. a square thereof. The system makes use of a constant value and various constants of proportionality that have been derived from a statistical analysis of historic patient data for a plurality of patients. The method of this aspect may include such a statistical analysis. The statistical analysis may for example comprise a multivariate linear regression analysis accounting for the different cardiac markers. Alternatively, the statistical analysis may be based on non-linear analysis, multiple logistic regression, discriminant analysis, or neural network analysis amongst others. The inventors have found that by making appropriate use of a suitably comprehensive dataset of a given patient population it is possible to estimate the minimum LV pressure via a statistical analysis, such as the multivariate linear regression described in more detail herein or alternative techniques based on the same cardiac markers.

As well as the markers discussed above, the cardiac markers used in the statistical analysis may also include other measures and/or estimates, such as one or more of the following: end systolic or end diastolic LV volume normalized to BMI or body surface area (respectively, ESVi or EDVi); the ratio between peak early diastolic velocity and peak late diastolic velocity across MV (EoverA); maximal mitral annular velocity at early diastole filling (e′) and/or LA volume indexed to body surface area (LAVi).

The systolic pressure, which can refer to arterial, aortic or LV systolic pressure, is a peak system pressure and can be measured via suitable means, such as via a blood pressure cuff. The method may include measuring the systolic pressure. The method may include using the data input device for input of systolic pressure data, e.g. as a numeric value. BMI can be obtained in known ways, i.e. from height and weight measurements and once again the method may include receiving this measure via the data input device. That may be done as the source height and weight data, or as input of a pre-calculated BMI. The imaging data is used to obtain an estimate of LA reservoir strain and an estimate of the time constant of LV isovolumic pressure decay. In this method we use a non-invasive imaging based method to obtain the time constant of LV isovolumic pressure decay. The same imaging data may also be used for the estimation of peak pressure drop at step (c).

The LA reservoir strain may be any suitable indicator of strain in this context, such as a long axis reservoir strain, a short axis reservoir strain, a mean area strain or a volumetric measure of strain.

The summation of step (b) may be considered as:

$$A + B \times ( systolic pressure ) + C \times ( BMI ) + D \times ( estimate of LA reservoir strain ) + E \times ( estimate of the time constant of LV isovolumic pressure decay )$$

where each of A, B, C, D and E are constants obtained via the statistical analysis. Optionally, there may be further terms in the sum for ESVi, EDVi, EoverA, e′, LV mass and/or LAVi as mentioned above, each with their own constant of proportionality.

In one example, the constant A is in the range −24.1 to 0.3, for example it may be in the range −14.4 to −6.5. Optionally it is about −6.9, for example −6.885.

In one example, the constant B, i.e. the constant of proportionality associated with the systolic artic pressure (peak system pressure) is in the range 0.001 to 0.056, for example it may be in the range 0.02 to 0.03. Optionally it is about 0.025.

In one example, the constant C, i.e. the constant of proportionality associated with the BMI is in the range −0.085 to 0.432, for example it may be in the range 0.10 to 0.30. Optionally it is about 0.243.

In one example, the constant D, i.e. the constant of proportionality associated with the estimate of LA reservoir strain is in the range −0.126 to 0.040, for example it may be in the range −0.10 to 0.03. Optionally it is about −0.0973.

In one example, the constant E, i.e. the constant of proportionality associated with the estimate of the time constant of LV isovolumic pressure decay is in the range 0.097 to 0.275, for example it may be in the range 0.12 to 0.20. Optionally it is about 0.1575.

Thus, in one particular example the summation of step (b) may be expressed as:

$$- 6.885 + 0.025 \times ( systolic pressure ) + 0.243 \times ( BMI ) - 0.0973 \times ( estimate of LA reservoir strain ) + 0.1575 \times ( estimate of the time constant of LV isovolumic pressure decay )$$

Where one or more of ESVi, EDVi, EoverA, e′, and/or LAVi are included then the relevant constants may be in the ranges tabulated below:

TABLE 2
Constant ranges for the potential inclusion of further
cardiac markers in the statistical analysis.

| Cardiac marker | Minimum | Maximum |
|---|---|---|
| ESVi | −0.049 | 0.046 |
| EDVi | −0.034 | 0.052 |
| EoverA | −2.828 | 1.658 |
| e′ | −0.172 | 0.724 |
| LAVi | −0.106 | 0.091 |

The method may include determining an estimated LV diastolic pressure curve showing estimated pressure throughout diastole. This may be done by obtaining key markers for LV pressure in relation to time and/or pressure for points in the cycle and fitting a normalised curve shape to the key markers. The key markers for the estimated LV pressure curve will include the estimated LV filling pressure as well as one or more of: time of aortic valve closure; time of MV opening; minimum LV pressure and respective time; time of peak E-wave; time of end diastasis, time of peak A-wave, and time of end diastole (corresponding to MV closure). In addition, the pressure may be estimated at some or all of these time points, such that the method may comprise a non-invasive estimate of at least seven patient-specific pressure-time points thus enabling close fitting of a normalised curve. The normalised curve may be an average of previously obtained sample patient data. The data processing apparatus may be configured to interpolate curve segments of the normalised curve in order to fit to the key markers of pressure and time to thereby obtain the estimated LV pressure curve. Furthermore, in some examples, the increase in LV pressure caused by LA contraction will be calculated from the estimated LA to LV pressure difference and estimated diastolic stiffness of the left ventricle.

The method may include non-invasively obtaining images of the patient's heart using an imaging system. The imaging system may be, for example, an ultrasound imaging system, a MRI system, an x-ray imaging system such as CT, or any other system able to obtain suitable images of the heart. In example embodiments echocardiography is used and hence the imaging system may comprise an ultrasound probe and associated computer devices as are already known for use in echocardiography. The method may be used as a part of a method for diagnosing and treating patients with HFpEF or other cardiac conditions, within which method the LV filling pressure of a patient is estimated using a method as in the third aspect. The patient with heart failure is determined to require treatment if the estimated LV filling pressure is equal to or in excess of 15 mmHg, optionally in excess of 12 mmHg, and if the patient is determined to require HFpEF treatment then a drug indicated for treatment of HFpEF is administered. The drug may for example be Empagliflozin, Entresto or another SGLT inhibitor.

Viewed from a still further aspect the invention provides an HFpEF drug for use as a medicament for treatment of HFpEF via a method as defined above. Thus, the invention may be embodied as a HFpEF drug for use as a medicament for treatment of HFpEF via a method for diagnosing and treating patients with HFpEF, within which method the LV filling pressure of a patient is estimated using a method as in the third aspect, the patient is determined to require treatment if the estimated LV filling pressure is in excess of 15 mmHg, optionally in excess of 12 mmHg, and if the patient is determined to require HFpEF treatment then a drug indicated for treatment of HFpEF is administered, such as Empagliflozin or Entresto. The invention may alternatively or additionally comprise a method of use of a HFpEF drug for the manufacture of a medicament for treatment of HFpEF via a method as defined above.

### BRIEF DESCRIPTION OF THE DRAWINGS

Certain example embodiments of the invention will now be described by way of example only and with reference to the accompanying drawings in which:

FIG. 1 is a diagram showing an estimated LV pressure curve;

FIG. 2 shows a comparison of invasively measured LV pressure and estimated LV pressure;

FIGS. 3 to 9 illustrate data for seven patients with comparisons of invasively measured LV pressure and estimated LV pressure, where the estimation uses echocardiograph data obtained simultaneously with the invasive pressure recordings; and

FIG. 10 is a schematic diagram of a system for estimating LV filling pressure.

### DETAILED DESCRIPTION

LV diastolic pressure can be measured as the average pressure during diastole, as pressure prior to atrial contraction (pre-A pressure), and as end-diastolic pressure, and all these pressures provide information of essentially similar clinical value.

Because left atrium and left ventricle are directly connected in diastole, and the left atrium is directly connected with the pulmonary vasculature, elevation of LV diastolic pressure is transmitted to the pulmonary vasculature. Thus, when LV diastolic pressure becomes elevated, pressure in the pulmonary capillaries increase with about similar magnitude. This leads to pulmonary congestion and breathing problems. Therefore, LV diastolic pressure is so important to know and to target when treating heart failure patients.

Direct measurement of LA pressure is not feasible clinically, but LV pre-A pressure is a good approximation of LA mean pressure. Therefore, LV pre-A pressure is an important parameter to measure. In the present innovation, LV pre-A pressure is employed as an indirect measure of LA mean pressure. There are rare cases with MV stenosis when this assumption is not valid. Pulmonary capillary wedge (PCWP) pressure measured during right heart catheterization, is used as an indirect measure of LA mean pressure.

In this document explicit mention of physical unit is sometimes omitted. Unless stated otherwise, all pressures are given in mmHg. Some equations show apparently incompatible units, for example equation (3) where ΔP_{A}≈4vₘₐₓ². It is understood that vₘₐₓ is given in m/s, and ΔP_{A} is in mmHg, while the calculation is carried out in a unit less fashion.

A basic assumption in our innovation is that LA mean pressure can be approximated by the sum of the minimum LV diastolic pressure, P_{LV}, and the peak LA to LV pressure drop during early diastole, P_{LA}−P_{LV}. This assumption has been supported by several studies in conscious and anaesthetized dogs under a wide range of hemodynamic conditions and by observations in patients.

$$〈 {P}_{LA} 〉 \approx min {P}_{LV} + max ( {P}_{LA} - {P}_{LV} ) \qquad ( 1 )$$

The method consists of three steps: estimation of the maximum pressure drop, estimation of the minimum LV pressure, and estimation of (P_{LA}) as detailed in the following sections.

The algorithm for calculating the maximum pressure drop, max(P_{LA}−P_{LV}), requires the inputs listed in Table 3 below.

TABLE 3
Measurements required for estimation
of maximal pressure drop across MV

| Symbol | Description | Units | Origin |
|---|---|---|---|
| [v_{MV}] | Flow velocity trace | m/s | Echocardiographic |
| | across the mitral valve | | examination (or other medical |
| | | | imaging modality) |
| | | | Echocardiographic |
| [Q] | Flow rate trace | ml/s | examination (or other medical |
| | across mitral valve | | imaging modality) |
| [Δ Q] | Change of flow | ml/s | Echocardiographic |
| | rate trace between | | examination (or other medical |
| | consecutive frames | | imaging modality) |

The pressure drop estimate is based on the Navier-Stokes momentum conservation equation:

$$Δ P = - \frac{1}{Q} ( \frac{\partial K}{\partial t} + A + V ) = Δ {P}_{A} + Δ {P}_{K} + Δ {P}_{V} \qquad ( 2 )$$

Where Q is the flow rate computed across the MV over the diastolic period, t is the temporal derivative of the kinetic energy within MV inflow jet, A is the advective energy rate describing the energy transfer because of the physical movement of a fluid in and out of the domain, and V is the rate of viscous dissipation describing energy losses because of friction.

The viscous term, ΔP_{V}, can be neglected in the case of relatively high velocities, such as in flow across the MV.

The advective term is approximated using the simplified Bernoulli equation:

$$Δ {P}_{A} \approx 4 {v}_{MV}^{2} \qquad ( 3 )$$

The kinetic term is calculated as:

$$Δ {P}_{K} \approx \frac{ρ {Qv}_{MV}^{2}}{2 Δ Q} \qquad ( 4 )$$

where the term ρ=1060 kg/m³ is the density of blood.

The time of minimum LV pressure, t_{min P}_{^{LV}}, can be approximated by the peak pressure drop during early diastole, which in turn is given by the time of peak E-acceleration, i.e. by the maximum value of the trace of the flow rate variation, ΔQ. Equations (3) and (4) are therefore evaluated at that time.

The resulting estimate for the pressure drop is:

$$max ( {P}_{LA} - {P}_{LV} ) \approx 4 {v}_{MV}^{2} ( {t}_{{minP}_{LV}} ) + \frac{ρ Q ( {t}_{{minP}_{LV}} ) {v}_{MV}^{2} ( {t}_{{minP}_{LV}} )}{2 Δ Q ( {t}_{{minP}_{LV}} )} \qquad ( 5 )$$

The algorithm for calculating the minimum LV pressure, min P_{LV}, requires the following inputs (Table 4 below). In some cases the algorithm can be adapted to use differing parameters/markers, such as by using height and/or weight directly in place of using BMI.

TABLE 4
Measured and estimated inputs for the
estimation of minimum LV pressure.

| Symbol | Description | Units | Origin |
|---|---|---|---|
| P_{sAO} | Systolic aortic | mmHg | Cuff measurement performed |
| | pressure | | by clinician |
| BMI | Body mass index | Unitless | Height and weight |
| | | | measurement |
| ε_{rLA} | LA reservoir strain | Unitless | Echocardiographic |
| | | | examination (or other |
| | | | medical imaging |
| | | | modality) |
| | | | Echocardiographic |
| | | | examination (or other |
| t_{AVC} | Time of aortic | Seconds | medical imaging |
| | valve closure | | modality) |
| | | | Echocardiographic |
| | | | examination (or other |
| t_{MVO} | Time of mitral | Seconds | medical imaging |
| | valve opening | | modality) |
| t_{max dP} | Time of maximum | Seconds | Echocardiographic |
| | LA-LV pressure drop | | examination (or other |
| | | | medical imaging |
| | | | modality) |

Statistical analysis of clinical data has shown that the min P_{LV} can be approximated by a regression on several other clinical parameters, so that min P_{LV} is almost equal to

$$P = {c}_{0} + {c}_{1} \cdot {P}_{sAO} + {c}_{2} \cdot BMI + {c}_{3} \cdot {ε}_{rLA} + {c}_{4} \cdot τ \qquad ( 6 )$$

The statistical analysis of the clinical data yielded the following as the best linear approximation:

$$min {P}_{LV} \approx min {P}_{LV} ( τ ) = - 6.885 + 0.025 \cdot {P}_{sAO} + 0.243 \cdot BMI - 0.0973 \cdot {ε}_{rLA} + 0.1575 \cdot τ \qquad ( 7 )$$

It is important to note that other regression coefficients can be used, also including further variables including end-diastolic volume (absolute or indexed to BSA), E/A transmitral velocity ratio, E/e′ velocity ratio, or e′ tissue velocity amongst other. The units of the clinical parameters are specified in Table 2, while the isovolumic relaxation time constant, τ, is given in seconds.

The calculation of min P_{LV} is presented as a function of t, since these are interdependent variables which require a numerical solver to find the roots (\hat{τ} and min P_{LV} (τ\hat{ })). The time constant t describes the rate of decay of the LV pressure during the period where both the LV valves, the aortic valve (AV) and the MV, are closed; that is, a time interval between the closing of the aortic valve, t_{AVC}, and the opening of the MV, t_{MVO}. During this time interval the LV pressure can be approximated by the expression

$${P}_{LV} ( t ) \approx {P}_{LV} ( {t}_{0} ) \cdot {e}^{- ( t - {t}_{0} ) / τ} for t in [ {t}_{0} , {t}_{1} ] \qquad ( 8 )$$

where t₀, t₁ are time instances during the isovolumic relaxation phase, P_{LV}(t) is the LV pressure at time t, P_{LV}(t₀) is the LV pressure at t₀. From this we get that

$$τ = \frac{{t}_{1} - {t}_{0}}{ln {P}_{LV} ( {t}_{0} ) - ln {P}_{LV} ( {t}_{1} )} \qquad ( 9 )$$

Where ln denotes the natural logarithmic function.

Several different choices for t₀ and t₁ are possible. One possible choice is

$${t}_{0} = {t}_{min dP / dt} \approx {t}_{AVC} \qquad ( 10 )$$

$${t}_{1} = {t}_{MVO}$$

Based on statistical analysis of a suitable dataset, the following approximations are used:

$${P}_{LV} ( {t}_{0} ) \approx 0.65 \cdot {P}_{sAO} \qquad ( 11 )$$

$${P}_{LV} ( {t}_{1} ) \approx {P}_{MVO} + 5 = min {P}_{LV} ( τ ) + max ( {P}_{LA} - {P}_{LV} ) + 10$$

The motivation for the approximation of P_{LV}(t₁) is to neglect the P_{LV} variation due to hemodynamic changes at the time of onset LV filling, P_{LV}(t₁) is set 5 mmHg above P_{MVO}, and P_{MVO} is defined as:

$${P}_{MVO} \approx 〈 {P}_{LA} 〉 + 5 mm Hg \qquad ( 12 )$$

Finally, in the proposed P_{LV} curve estimation, the IVR has an exponential behavior down to P_{MVO}+5 mmHg and a linear behavior afterwards.

Values for min P_{LV} and τ can be obtained by combining equations (7) and (9) and solving for the unknown quantities. For example, substituting the right-hand side of (9) for τ in (7) results in the following equation:

$$f ( τ ) = τ \cdot ( b - ln ( min {P}_{LV} ( τ ) + c ) ) - Δ t = 0 \qquad ( 13 )$$

Where

$$Δ t = {t}_{min dP / dt} - {t}_{MVO}$$

$$b = 0.65 {P}_{sAO}$$

$$c = 4 {v}_{max}^{2} + \frac{ρ {Qv}_{max}^{2}}{2 Δ Q} + 10$$

$$min {P}_{LV} ( τ ) = - 6.885 + 0.025 \cdot {P}_{sAO} + 0.243 \cdot BMI - 0.0973 \cdot {ε}_{rLA} + 0.1575 \cdot τ$$

The estimated value, τ\hat{ }, for the isovolumic relaxation time constant, τ, is found by numerically solving equation (13). This can be done with any standard root-finding software, and an initial guess for the value of τ\hat{ }. The initial guess for the value of τ\hat{ }can e.g., be set by inserting an initial value of P_{LV}(t₁), following equation (11), into equation (9), one example of this being 20 mmHg.

After finding the root, τ\hat{ }, of equation (13) the LV filling pressure described in equation (1) can be presented as the sum of equation (7) in function of τ\hat{ } and equation (5):

$$〈 {P}_{LA} 〉 = min {P}_{LV} ( \hat{τ} ) + 4 {v}_{MV}^{2} ( {t}_{{minP}_{LV}} ) + \frac{ρ Q ( {t}_{{minP}_{LV}} ) {v}_{MV}^{2} ( {t}_{{minP}_{LV}} )}{2 Δ Q ( {t}_{{minP}_{LV}} )} \qquad ( 14 )$$

The LA contraction induced P_{LV} rise accounts for three independent P_{LV} variations that are computed over two periods of atrial contraction:
From pre-A to peak-A wave velocity, P_{LV} rise is computed as the sum of pressure increase due to passive stiffness together with the maximal MV dP during atrial contraction. The passive stiffness is derived from the LV pressure-volume relationship that rules diastasis in our method (including estimation of P_{LV} from peak E-wave).

The maximal MV dP is obtained as the sum of the convective and kinetic components at the instant of maximal A-wave acceleration. The A-acceleration is calculated from transmitral velocities by pulsed Doppler. The A-wave upslope can be approximated to a sinusoidal behavior since at the beginning of atrial contraction the acceleration is lower, the maximal acceleration is reached at the middle of A-wave upslope and then the acceleration is reduced until the peak A velocity is reached. Inherently, the maximal A-acceleration is considered to occur at ½ of A-wave acceleration duration, and the velocity at this instant is also ½ of the peak A velocity.

Furthermore, the peak A-acceleration is given by the mean A-acceleration slope multiplied by the acceleration constant: K_{acc}=PI/2˜1.57. This constant K_{acc} is obtained from the relationship between the maximal and mean derivative of any sinusoidal upslope trace. This value is also very similar to the value found experimentally across the current cohort when dividing maximal A-acceleration by mean A-acceleration.
From peak-A to MVC, the pressure change is estimated by LV compliance and LV volume during A-wave deceleration. We consider a constant compliance/stiffness behavior during A-wave deceleration. In line with this assumption, we consider a linear decrease in velocity (constant deceleration) given by the peak A-wave velocity (minus ED trans-MV velocity which is considered to be zero) divided by the A deceleration time. The A deceleration time is obtained via trans-MV Doppler or from the end-diastolic strain trace (from peak A strain rate to MVC). Also, from the volume/strain trace is obtained the change in volume during the same period. The effective orifice area of the A-wave is computed. Finally, we can apply equations (15) and (16) of compliance calculation to compute the change in pressure during this period:

$${C}_{n} = - \frac{A}{ρ \cdot dv / dt} \qquad ( 15 )$$

$${C}_{n} = - \frac{dV / dt}{dp / dt} \qquad ( 16 )$$

resulting in the constant change in pressure of: dP/dt=(dV/dt)/Cn. The equation (15), considering that all relations are constant, can be extrapolated to:

$$\frac{Δ P}{Δ t} = - \frac{Δ V / Δ t}{{C}_{n}} \qquad ( 17 )$$

Finally, the EDP is obtained as the sum of A-deceleration ΔP with the peak-A P_{LV}.

In order to validate the atrial contraction induced P_{LV}, a dataset of n=7 patients undergoing simultaneously echocardiography and catheterization examination were considered. The results show a good agreement between measured and non-invasive estimative of atrial contraction induced P_{LV} increase, as well as a good agreement for the estimation of end-diastolic P_{LV}. The results are presented in Table 5 below.

TABLE 5
Case-by-case comparison between simultaneously measured
and estimated LV pressure (P_{LV}) due to atrial contraction
at peak A-wave and mitral valve closure (MVC) instances

| | Case # | 1 | 2 | 3 | 4 | 6 | 7 | 8 | mean | std dev. |
|---|---|---|---|---|---|---|---|---|---|---|
| Peak A- | Measured | 12.5 | 32.9 | 17.4 | 9.0 | 8.1 | 9.5 | 20.1 | 15.8 | 8.1 |
| wave P_{LV} | Estimated | 15.6 | 29.5 | 15.7 | 9.4 | 7.8 | 12.4 | 15.2 | 15.1 | 6.6 |
| MVC P_{LV} | Measured | 11.5 | 32.2 | 17.4 | 9.0 | 9.8 | 9.5 | 16.5 | 15.1 | 7.6 |
| | Estimated | 16.4 | 29.9 | 16.0 | 10.0 | 8.2 | 12.7 | 15.4 | 15.5 | 6.5 |

The final step of the provided methodology is the generation of the fully non-invasive and patient specific LV diastolic pressure curve. This curve is tabulated based on a reference curve for LV diastolic pressure, previously generated from data collected by Smiseth et al (1998). The method uses the calculated times and pressures for a set of key events during diastole, and subsequently fits the reference curve to those events using a combination of scaling and interpolation.

The reference LV diastolic pressure curve was collected invasively from several heart patients. The duration between each event was normalized, by interpolating each subsection of each recorded pressure curve onto a fixed set of time samples, and then averaging the pressure curves across all patients.

After normalization, the reference curve is specified as a sequence of values along with labels pointing to key events.

The following inputs are used to calculate the patient specific diastolic LV pressure curve. They consist of the times and pressures of several diastolic events, in addition to the reference pressure curve, sampled at a larger number of time points. Other events, more or fewer, can be used. The units of the reference pressure curve, P_{ref}, is given as “arbitrary” or “unitless”.

TABLE 6
Input arguments to algorithm of LV pressure curve

| Symbols | Description | Units |
|---|---|---|
| [Pₑᵥₜ] | List of pressures at | Arbitrary/ |
| | key diastolic events | unitless |
| [tₑᵥₜ] | List of times of | Seconds/ |
| | key diastolic events | time unit |
| [P_{ref}] | List of reference pressures, | Arbitrary/ |
| | including among them the | unitless |
| | pressures at the key diastolic events. | |
| [indₑᵥₜ] | Sequence of sample numbers in | Unitless |
| | [P_{ref}] at which | |
| | key diastolic events occur | |

For each time range between two events, the estimated patient specific curve is fitted to the corresponding samples from the reference curve, P_{ref}. In order to accomplish this, both the sample times and the sample pressure values can be interpolated onto the reference curve; for example, to calculate time- and pressure-sample number k, respectively equations (18) and (19), for samples occurring between the first and second key event (evt):

$${t}_{k} = {t}_{evt} ( 1 ) + \frac{k - {ind}_{evt} ( 1 )}{{ind}_{evt} ( 2 ) - {ind}_{evt} ( 1 )} \cdot ( {t}_{evt} ( 2 ) - {t}_{evt} ( 1 ) ) \qquad ( 18 )$$

$${P}_{k} = {P}_{evt} ( 1 ) + \frac{{P}_{ref} ( k ) - {P}_{ref} ( {ind}_{evt} ( 1 ) )}{{P}_{ref} ( {ind}_{evt} ( 2 ) ) - {P}_{ref} ( {ind}_{evt} ( 1 ) )} \cdot ( {P}_{evt} ( 2 ) - {P}_{evt} ( 1 ) ) \qquad ( 19 )$$

This procedure is repeated for the pressure curve segments between each pair of key events.

A specific implementation of this algorithm, requires a set of diastolic markers to serve as the key events. For example, the events listed in Table 7 can be used.

TABLE 7
Diastolic markers used for a specific implementation
of the LV pressure curve construction algorithm.
Other events, more of fewer can be used.

| Symbols | Description | Units |
|---|---|---|
| (P_{AVC}, t_{AVC}) | Pressure and time at | mmHg/seconds |
| | aortic valve closure | |
| (P_{MVO}, t_{MVO}) | Pressure and time at | mmHg/seconds |
| | mitral valve opening | |
| (min P_{LV}, t_{minLVP}) | Minimum left ventricular | mmHg/seconds |
| | pressure, and timing | |
| (P_{E-wave}, t_{E-wave}) | Pressure and time at end | mmHg/seconds |
| | mitral E-wave velocity | |
| (P_{A-wave-start}, | Pressure and time at start | mmHg/seconds |
| t_{A-wave-start}) | of A-wave velocity | |
| (P_{A-wave-peak}, | Pressure and time at peak | mmHg/seconds |
| t_{A-wave-peak}) | of A-wave velocity | |
| (P_{MVC}, t_{MVC}) | Pressure and time at | mmHg/seconds |
| | mitral valve closure | |

In order to calculate pressures and times listed in Table 4, the measured systolic P_{sAO} and the LV volume and blood flow traces are used as input parameters (Table 8).

TABLE 8
Input parameters used for calculating
the diastolic markers in Table 4.

| Symbol | Description | Units | Origin |
|---|---|---|---|
| [v_{MV}] | Flow velocity | m/s | Echocardiographic |
| | trace across | | examination (or other |
| | the mitral valve | | medical imaging modality) |
| [V] | Volume trace of LV | ml | Echocardiographic |
| | | | examination (or other |
| | | | medical imaging modality) |
| [Q] | Flow rate trace | ml/s | Echocardiographic |
| | across mitral | | examination (or other |
| | valve | | medical imaging modality) |
| P_{sLV} | Systolic LV pressure | mmHg | Cuff measurement, assumed |
| | | | equal to P_{sAo} in cases |
| | | | without aortic stenosis. |

Quantities V and Q are related through temporal differentiation, Q=dV/dt, so one can be obtained from the other, but because V and Q are often obtained from different measurements, they are considered separate inputs in this case. The quantities dV/dt and dQ/dt, used below, can be obtained through any standard numerical differentiation method.

Calculate (P_{AVC},t_{AVC}): The estimates are P_{AVC}≈0.65·max(P_{sLV}) where P_{sLV} is assumed to be equal to P_{sAO} in cases without aortic stenosis, and t_{AVC} is approximately the time of the negative peak of the [dP/dt] trace applied in the estimation of isovolumic relaxation time constant, τ, as described in equation (9).

Calculate (P_{MVO},t_{MVO}): The estimates of P_{MVO} are given by equation (12), and t_{MVO} is the first time point after t_{AVC} when both the volume trace is increasing and transmitral dQ/dt is positive.

Calculate (min P_{LV}, t_{min P}_{^{LV}}): The estimate of min P_{LV} is given in Equation (7). The estimate of t_{min P}_{^{LV}} is the time point of peak dQ/dt within the range of 45-80% of the time interval between t_{MVO} and t_{E-wave}. The time range was determined by combining data collected from various suitable sources.

Calculate (P_{E-wave}, t_{E-wave}): The estimate of P_{E-wave} is based on the end-diastolic pressure-volume relationship that characterizes ventricle stiffness, for example it can be assumed to be linear from E-wave peak velocity until A-wave-start: P_{E-wave}≈a·V(t_{E-wave})+b, and t_{E-wave} is the time of end of the deceleration of flow rate dQ/dt. The stiffness constants (a, b) are computed based on the estimated incomplete relaxation P_{LV}. Exemplary values are a=0.19, b=−8.08.

Calculate (P_{A}_{^{−wave-start}},t_{A}_{^{−wave-start}}): The estimates are P_{A}_{^{−wave-start}}≈ [symbol] P_{LA} [symbol] , and t_{A}_{^{−wave-start}} is the first time point after diastasis, when dV/dt and dQ/dt are both positive.

Calculate (P_{A}_{^{−wave-peak}}, t_{A}_{^{−wave-peak}}): The estimate of

$${P}_{A - wave - peak} = {P}_{A - wave - start} + {\int }_{{t}_{A - wave - peak}}^{{t}_{A - wave - start}} ( \frac{max ( {P}_{LA} - {P}_{LV} )}{dt} ) dt .$$

The maximal MV pressure difference max(P_{LA}−P_{LV}) is given by equation (5), at the time when A-wave acceleration is maximum (t_{A-wave-max-acceleration}≈(t_{A}_{^{−wave-start}}+t_{MVC})/2), mirroring for the A-wave the concepts applied for [symbol] P_{LA} [symbol] computation. t_{A}_{^{−wave-peak}} is the time of the peak late diastolic flow rate.

Calculate (P_{MVC},t_{MVC}): The pressure at end-diastole/MV closure is estimated based on the LV atrioventricular compliance across MV (Flachskampf 1992) during A-wave deceleration as P_{MVC}=P_{A-wave-peak}+(V−(t_{MVC})−V(t_{A-wave-peak}))/Cn, where the compliance is given by Cn=Area_{MV}/(ρ·(v_{MV}(t_{A-wa-e-peak})−v_{MV}(t_{MVC}) and effective orifice MV area during A-wave is Area_{MV}=Q(t_{A-wave-peak})/v_{MV}(t_{A-wave-peak}). t_{MVC} is at the end of the diastolic volume curve, when dV/dt is close to zero, i.e., the volume curve is flat.

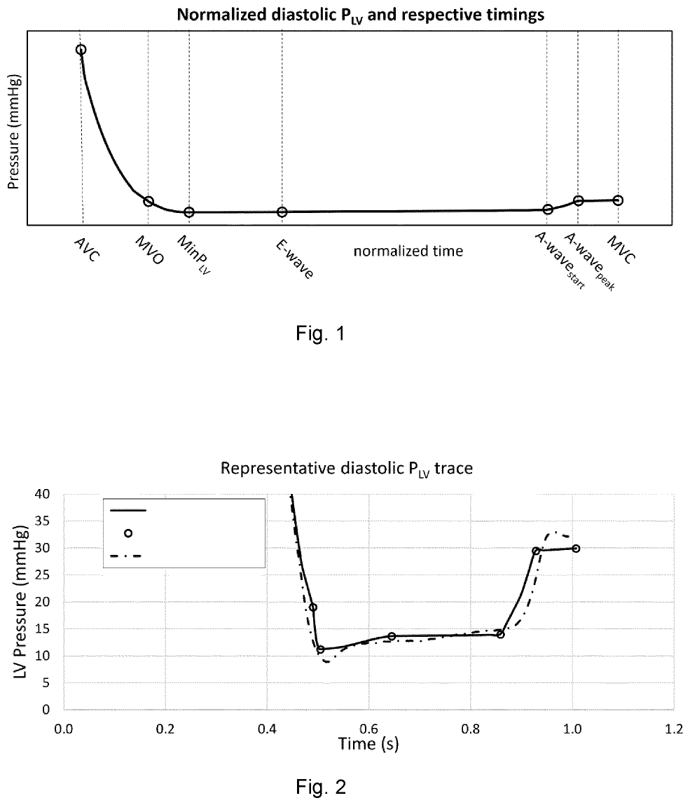

An example estimated patient specific curve is displayed in FIG. 1.

In order to validate the full diastolic P_{LV} curve, a dataset of n=7 patients undergoing simultaneously echocardiography and catheterization examination were considered. The results show a very good agreement between measured and non-invasive P_{LV} curves, both visually (FIG. 2), as well as quantitatively by measuring the mean diastolic P_{LV} from t_{MVO} to t_{MVC} (FIG. 2 and Table 9 below).

TABLE 9
Case-by-case comparison between simultaneously
measured and estimated mean diastolic P_{LV}.

| | | Case # | | | | | | | | std |
|---|---|---|---|---|---|---|---|---|---|---|
| | | 1 | 2 | 3 | 4 | 6 | 7 | 8 | mean | dev. |
| Mean | Measured | 6.0 | 15.7 | 10.6 | 2.7 | 3.7 | 4.7 | 12.4 | 8.0 | 4.6 |
| PLV | Estimated | 7.1 | 14.5 | 10.9 | 5.7 | 3.6 | 5.6 | 9.8 | 8.2 | 3.5 |

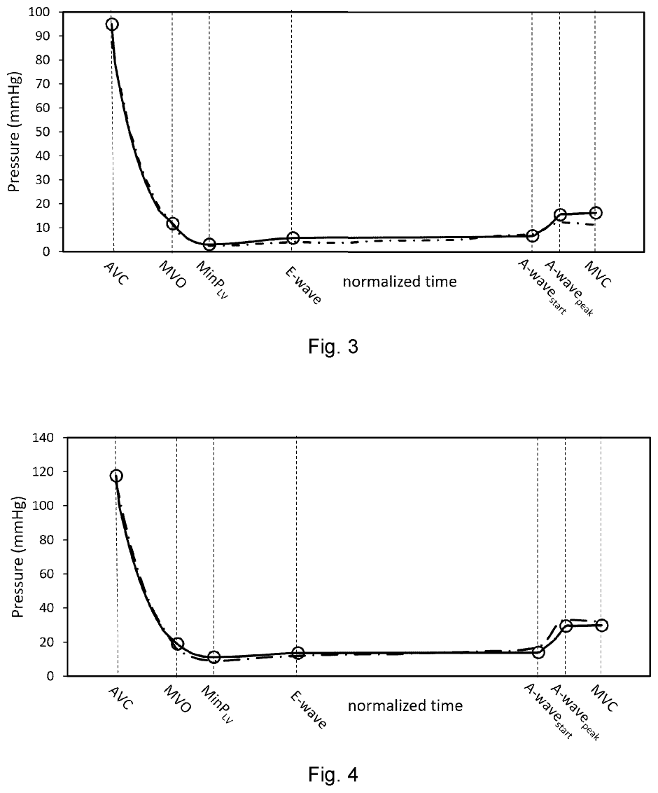

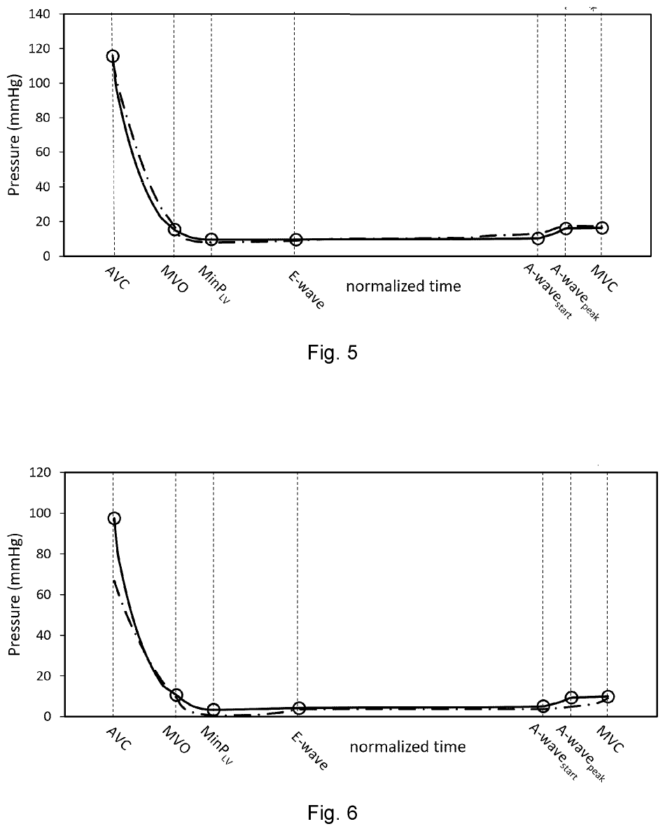

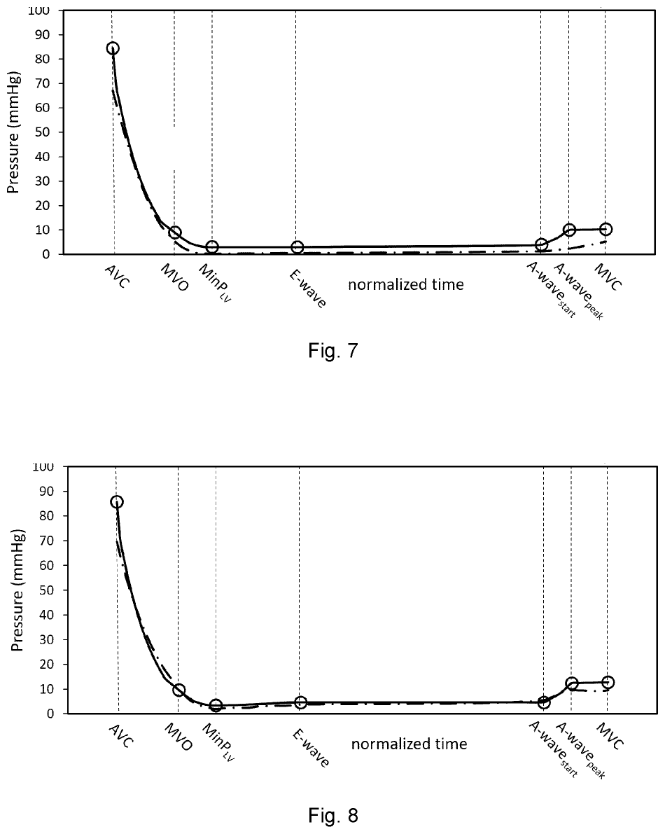

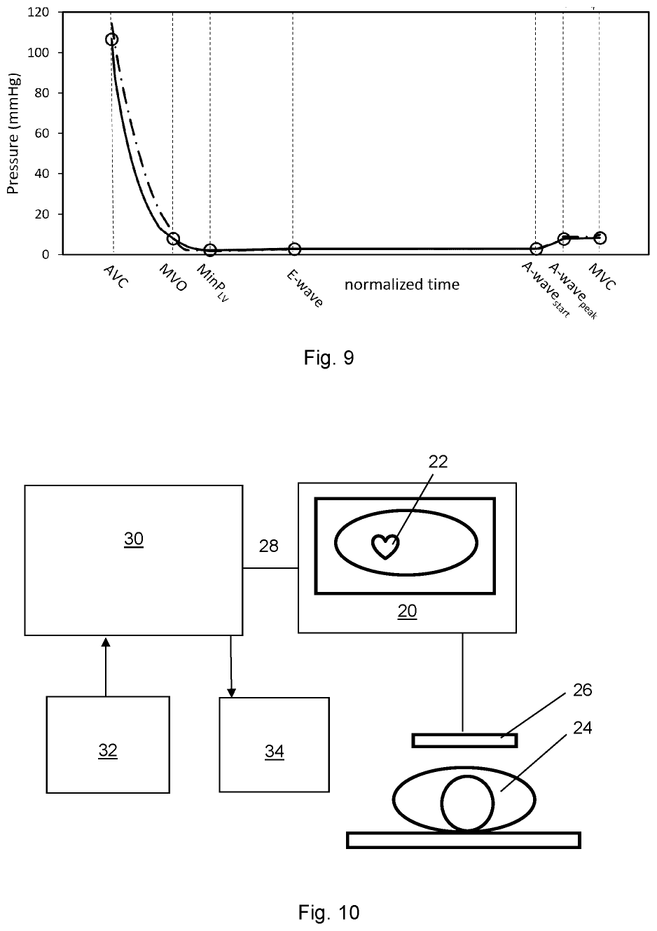

FIGS. 3 to 9 show Non-invasive diastolic LV pressure curves (blue trace) and markers (orange) in comparison with the respective measured pressure curves in n=7 patients, where the echocardiography acquisition was performed simultaneously with the invasive pressure recordings. The pressure traces are normalized over time to highlight the estimation of the different pressure markers. The diastolic LV pressure markers (estimates in orange) include: Aortic valve closure (AVC), Mitral valve opening (MVO), minimal LV pressure (Min P_{LV}), peak E-wave, LV pre-A pressure (A-waveₛₜₐᵣₜ), peak A-wave (A-waveₚₑₐₖ), and end diastole corresponding to the MV closure (MVC).

One possible product implementation is a software product that will provide a measurement of LV filling pressure as LA mean pressure and display/save/export the image of the LV diastolic pressure curve. The software product might be integrated with a commercial ultrasound scanner such that some input parameters do not need to be entered, or a post-processing tool that loads stored examination data.

In FIG. 10 an example of a possible system for estimating the LV filling pressure of the heart is shown in schematic form. The system comprises an imaging system 20 for non-invasively imaging the heart 22 of a human patient 24. The imaging system 20 may for example comprise an ultrasound probe 26, allowing for images to be obtained by echocardiography. The imaging system 20 provides imaging data 28 to a data processing apparatus 30, which may for example be a computer linked to the imaging system 20 or provided as an integrated part thereof. The data processing apparatus 30 is connected to a data input device 32 for receiving patient specific parameters. The data input device 32 may for example be configured to allow input of numeric data. The data processing apparatus 30 is also connected to a display device 34 for providing feedback to a user, such as by providing an indication of the estimated LV filling pressure, or an indication of whether this is above or below a threshold value. When the system is also able to determine an estimated pressure curve of the type shown in FIGS. 1 to 9 then the display device 34 may be used for displaying the estimated curve. Although the display device 34 and data input device 32 are shown separately they may be provided by a single device, such as a GUI or other peripheral(s) of the data processing device 30, or by an external computer device in communication with the data processing device 30. Such an external computer device may take the form of a tablet or smartphone, for example, including both the data input and display functions.

As summarized in the most recent guidelines on imaging of LV diastolic function from the Association of Cardiovascular Imaging in the European Society of Cardiology, there is currently no clinically applicable non-invasive method to measure LV filling pressure. Our innovation is the first non-invasive method to provide an estimate of LV filling pressure with good accuracy and which provides a measure of the LV diastolic pressure curve.

The main applications of this innovation will be to improve diagnosis of heart disease and to provide filling pressure as a guide when making therapeutic decisions.
1. To diagnose congestive heart failure: About 50% of all heart failure patients have normal LV ejection fraction (EF). In these patients the most important diagnostic criterion for heart failure is elevated LV filling pressure. The present innovation is a method to estimate LV filling pressure, and thereby to make the heart failure diagnosis in this large group of patients.
2. To guide heart failure therapy: Since heart failure symptoms are directly related to filling pressure, the invention may be used to guide heart failure therapy.
   An important reason for hospitalization of heart failure patients, is breathing problems due to elevated LV filling pressure which leads to pulmonary congestion. The present innovation may be used to assess filling pressure and to institute therapy prior to pulmonary congestion, and thereby avoid hospitalization.
3. To identify heart disease in patients with unclear diagnosis: Elevated LV filling pressure is a compensatory mechanism in many heart disorders, which may be used as objective sign of heart disease. This includes patients with atypical symptoms, patients in the emergency department with chest pain of unknown etiology and others. In patients hospitalized acutely with suspected myocardial infarction. demonstration of elevated LV filling pressure confirms there is a cardiac malfunction, which in turn can accelerate patient referral for coronary angiography.
4. To determine timing of heart valve surgery: The finding of elevated LV filling pressure indicates that the valve dysfunction has negative impact on the heart and is one of the criteria considered when deciding the timing of valve surgery.
5. To differentiate between pulmonary arterial hypertension and pulmonary hypertension secondary to elevated LA pressure: This important differentiation is currently feasible only with invasive pressure measurements.

It should be apparent that the foregoing relates only to the preferred embodiments of the present application and the resultant patent. Numerous changes and modification may be made herein by one of ordinary skill in the art without departing from the general spirit and scope of the invention as defined by the following claims and the equivalents thereof.

We claim:

1. A system for estimating LV filling pressure of the heart of a human patient, the system comprising:
a data processing apparatus;
an imaging system for non-invasively obtaining images of the patient's heart and for providing imaging data to the data processing apparatus; and
a data input device for receiving patient specific parameters relating to BMI and systolic arterial blood pressure of the patient and for providing the patient specific parameters to the data processing apparatus;
wherein the data processing apparatus is configured to:
(a) use the imaging data to obtain cardiac markers including an estimate of LA reservoir strain and an estimate of the time constant of LV isovolumic pressure decay, and use the patient specific parameters directly or to determine additional cardiac markers including systolic pressure and BMI;
(b) determine an estimate of the minimum LV diastolic pressure as a summation of a value derived from each of the cardiac markers multiplied by a corresponding constant of proportionality and added to a constant value, wherein the constant value and the various constants of proportionality have been derived from a statistical analysis of historic patient data for a plurality of patients;
(c) estimate the peak pressure drop during early diastole between left atrium over left ventricle and hence obtaining a maximum difference between the LA pressure and LV pressure; and
(d) calculate an estimate of LV filling pressure as the sum of values from step (b) and step (c) above.

2. A system as claimed in claim 1, wherein the data processing apparatus is configured to compare the estimate of LV filling pressure to a threshold value.

3. A system as claimed in claim 1, wherein the threshold value is used to assess if the estimate of LV filling pressure is equal to or in excess of 15 mmHg, or optionally in excess of 12 mmHg.

4. A system as claimed in claim 1, wherein the summation of step (b) is considered as:
$$A + B \times ( systolic pressure ) + C \times ( BMI ) + D \times ( estimate of LA reservoir strain ) + E \times ( estimate of the time constant of LV isovolumic pressure decay )$$
wherein each of A, B, C, D and E are constants obtained via the statistical analysis of the historic patient data; and
wherein one or more of the following applies:
the constant A is in the range −24.1 to 0.3;
the constant B is in the range 0.001 to 0.056;
the constant C is in the range −0.085 to 0.432;
the constant D is in the range −0.126 to 0.040; and/or
the constant E is in the range 0.097 to 0.275.

5. A system as claimed in claim 4, wherein at least one of the following applies:
the constant A is about −6.9;
the constant B is about 0.025;
the constant C is about 0.243;
the constant D is about −0.0973; and/or
the constant E is about 0.1575.

6. A system as claimed in claim 1, wherein the summation of step (b) is expressed as:
$$- 6.885 + 0.025 \times ( systolic pressure ) + 0.243 \times ( BMI ) - 0.0973 \times ( estimate of LA reservoir strain ) + 0.1575 \times ( estimate of the time constant of LV isovolumic pressure decay ) .$$

7. A system as claimed in claim 1, wherein the data processing apparatus is configured to provide an estimated LV diastolic pressure curve showing estimated pressure throughout diastole by obtaining key markers for LV pressure in relation to time and/or pressure for points in the cycle and fitting a normalised curve shape to the key markers, wherein the normalised curve is an average of previously obtained sample patient data.

8. A system as claimed in claim 7, wherein the key markers for the estimated LV pressure curve include the estimated LV filling pressure as well as one or more time points selected from: time of aortic valve closure; time of MV opening; minimum LV pressure and respective time; time of peak E-wave; time of end diastasis, time of peak A-wave, and time of end diastole.

9. A system as claimed in claim 8, wherein the blood pressure is estimated at some or all of the time points, such that the system is configured to provide a non-invasive estimate of up to seven patient-specific pressure-time points.

10. A system as claimed in claim 9, wherein the data processing apparatus is configured to interpolate curve segments of the normalised curve in order to fit to the key markers of pressure and time to thereby obtain the estimated LV pressure curve.

11. A system as claimed in claim 7, wherein the increase in LV pressure caused by LA contraction will be calculated from the estimated LA to LV pressure difference and estimated diastolic stiffness of the left ventricle.

12. A system as claimed in claim 1, wherein the imaging system comprises one of: an ultrasound imaging system, a MRI system, an x-ray imaging system such as CT, or any other system able to obtain suitable images of the heart.

13. A computer programme product comprising instructions, which when executed on a data processing apparatus will configure the data processing apparatus to receive imaging data from an imaging system; receive patient specific parameters relating to BMI and systolic arterial blood pressure of the patient; and then:
(a) use the imaging data to obtain cardiac markers including an estimate of LV reservoir strain and an estimate of the time constant of LV isovolumic pressure decay, and use the patient specific parameters directly or to determine additional cardiac markers including systolic pressure and BMI;
(b) determine an estimate of the minimum LV diastolic pressure as a summation of a value derived from each of the cardiac markers multiplied by a corresponding constant of proportionality and added to a constant value, wherein the constant value and the various constants of proportionality have been derived from a statistical analysis of historic patient data for a plurality of patients;
(c) estimate the peak pressure drop during early diastole between left atrium over left ventricle and hence obtaining a maximum difference between the LA pressure and LV pressure; and
(d) calculate an estimate of LV filling pressure as the sum of values from step (b) and step (c) above.

14. A method of estimating LV filling pressure of the heart of a human patient, the method comprising:
(a) using non-invasive imaging and other non-invasive measurement of the patient, determining cardiac markers including: systolic pressure, BMI or other patent specific parameters such as height and weight, an estimate of LA reservoir strain and an estimate of the time constant of LV isovolumic pressure decay;
(b) determining an estimate of the minimum LV diastolic pressure as a summation of a value derived from each of the cardiac markers multiplied by a corresponding constant of proportionality in addition to a constant value, wherein the constant value and the various constants of proportionality have been derived from statistical analysis of historic patient data for a plurality of patients;
(c) estimating the peak pressure drop during early diastole between left atrium over left ventricle and hence obtaining a maximum difference between the LA pressure and LV pressure; and
(d) calculating an estimate of LV filling pressure as the sum of values from step (b) and step (c) above.

15. A method for diagnosing and treating patients with HFpEF and no other specific etiology of heart disease identified, the method comprising: estimating LV filling pressure of a patient as claimed in claim 14, determining that the patient requires HFpEF treatment if the estimated LV filling pressure is equal to or in excess of 15 mmHg, optionally in excess of 12 mmHg, and if the patient is determined to require HFpEF treatment then a drug indicated for treatment of HFpEF is administered.

16. An HFpEF drug for use as a medicament for treatment of HFpEF in a method for diagnosing and treating patients with HFpEF as claimed in claim 15.

17. A method as in claim 16 wherein the drug is Empagliflozin, Entresto, or another SGLT inhibitor.
